(19) United States
(12) Patent Application Publication
(10) Pub. No.: US 2025/0009614 A1
(43) Pub. Date: Jan. 9, 2025

(54) ALCOHOL-FREE ANTIMICROBIAL HAND SANITIZER

(71) Applicant: COUNCIL FOR SCIENTIFIC AND INDUSTRIAL RESEARCH, Pretoria (ZA)

(72) Inventors: Tshepo Patric NKUNA, Pretoria (ZA); Michel Lonji KALOMBO, Pretoria (ZA); Katlego Zebedius SETSHEDI, Pretoria (ZA)

(73) Assignee: COUNCIL FOR SCIENTIFIC AND INDUSTRIAL RESEARCH, Pretoria (ZA)

(21) Appl. No.: 18/711,560

(22) PCT Filed: Nov. 2, 2022
(86) PCT No.: PCT/IB2022/060540

§ 371 Date: May 17, 2024

(30) Foreign Application Priority Data
Nov. 17, 2021 (ZA) 2021/09136

(51) Int. Cl. A61K 8/06; A61Q 17/00

(52) U.S. Cl. CPC A61K 8/06; A61Q 17/005; A61K 2800/21; A61K 2800/30; A61K 2800/524; A61K 2800/594

(57) ABSTRACT

The current invention relates to a method for producing an alcohol-free, antimicrobial, moisturising hand sanitizer comprising (i) a polymer-based nanoemulsion including one or more active compounds selected from cyanovirin-N (CV-N), cannabidiol (CBD) and palmitic acid (PA); and (ii) an aqueous gel-like moisturizing composition comprising xanthan gum, glycerol and a preservative such as citric acid. The invention further relates to an alcohol-free, antimicrobial, moisturising hand sanitizer produced by the method of the invention, as well as to use of the hand sanitizer as an antimicrobial, in particular an antiviral agent.

### FIELD OF THE INVENTION

The current invention relates to a method for producing an alcohol-free, antimicrobial, moisturising hand sanitizer comprising (i) a polymer-based nanoemulsion including one or more active compounds selected from cyanovirin-N (CV-N), cannabidiol (CBD) and palmitic acid (PA); and (ii) an aqueous gel-like moisturizing composition comprising xanthan gum, glycerol and a preservative such as citric acid. The invention further relates to an alcohol-free, antimicrobial, moisturising hand sanitizer produced by the method of the invention, as well as to use of the hand sanitizer as an antimicrobial, in particular an antiviral agent.

### BACKGROUND OF THE INVENTION

The global pandemic of the unprecedented Coronavirus disease-2019 (COVID-19), a highly infectious and deadly respiratory disease caused by the Severe Acute Respiratory Syndrome Coronavirus-2 (SARS-CoV-2) continues to afflict humanity. The virus is transmitted from human-to-human through direct contact with bodily fluids from infected individuals and mostly, aerosols (fomites) from coughing and sneezing. Recent studies have further shown that the virus can remain viable and infectious externally on various surfaces for up to 9 days (Jing et al., 2020). Wearing face masks and practicing good personal hand hygiene is of utmost importance to curb the spread of the disease and both mitigations are recommended by the World Health Organisation (WHO). Also, the WHO highly recommends the use of alcohol-based hand sanitizers (ABHS) as they are rapid in activity and have a broad spectrum of antimicrobial activity including viruses.

However, concerns have long been raised regarding the adverse health and safety hazards related to the high alcohol content required for maximum activity. The hazards are due to the chemical properties of alcohols; they are highly flammable and readily evaporate even at room temperatures (20-22° C.). The major concerns are the fire hazards they pose and how easily the vapors can cause explosions when mixed with air and an ignition source present in the near vicinity. These concentrations can also cause dizziness in ill-ventilated environments and irritations to the eyes and skin. According to the South African National Fire Protection Association (NFPA) classifications on flammable and combustible liquids, alcohols are Class 1 flammable liquids (highest class). Amongst potential health hazards is the life-threatening acute alcohol poisoning upon ingestion and affects children mostly. It is worth mentioning that the alcohol in beverages (ethyl alcohol) is not always the alcohol in sanitizers as isopropyl alcohol is also used and not suitable for ingesting.

Furthermore, the outermost layer of the skin consists of various lipids that form a protective barrier together with the skin flora (microbiome). Excessive and frequent use of alcohol-based sanitizers can dissolve and remove the lipid barrier and also degrade the skin flora. As a result, the skin becomes vulnerable and susceptible for secondary infections by other pathogens and leaves the skin dry. Dry and dehydrated skin can result in irritations and most likely to cause itching, flaking, scaling or peeling, cracks, and grey/ashy skin colour. Also, the removal of these barriers can cause painful inflammations (swelling) leaving the skin red and increases the likelihood of acquiring atopic dermatitis (eczema).

These risks and their potential harmful outcomes necessitate investigations in developing safer non-alcohol hand sanitizers such as alcohol-free hand sanitizers (AFHS) that are safe on the hands and efficacious, specifically against easily transmittable viruses such as the SARS-CoV-2.

To date, various methods for producing AFHS formulations have been developed for multiple personal hygiene practices. These technologies entail the use of benzalkonium chloride (BAC) or triclosan (TCS) as active antimicrobial agents with various thickeners, preservatives, and fragrances for application into many pharmaceuticals and personal care products such as medicated soaps, hand sanitizers, deodorants, toothpaste, air fresheners, and other cosmetic products. However, concerns regarding the safety of these antimicrobial agents have also been raised particularly TCS, which is currently being considered as an emerging contaminant.

There is therefore a need for a hand sanitizer that is safe and contains naturally occurring compounds having antimicrobial properties, and in particular antiviral properties that can be delivered efficiently.

### SUMMARY OF THE INVENTION

According to a first aspect of the invention, there is provided a process for producing an alcohol-free, antimicrobial, moisturising hand sanitizer, comprising the steps of:
(i) producing a polymer-based nanoemulsion comprising the steps of:
   A.I. co-dissolving at least one hydrophobic active compound selected from cannabidiol (CBD) and palmitic acid (PA), and at least one copolymer in a polar aprotic solvent;
   A.II. adding a surfactant to form an organic phase;
   A.III. dispensing the organic phase into an aqueous mixture comprising at least one hydrophilic polymer to form a nanoemulsion; and
   A.IV. stabilising the nanoemulsion by stirring, or
   B.I. dissolving at least one copolymer in a polar aprotic solvent;
   B.II. adding a surfactant to form an organic phase;
   B.III. dispensing the organic phase into an aqueous mixture comprising at least one hydrophilic polymer and at least one hydrophilic active compound selected from cyanovirin-N (CV-N) to form a nanoemulsion; and
   B.IV. stabilising the nanoemulsion by stirring, or
   C.I. co-dissolving at least one hydrophobic active compound selected from CBD, PA or both, and at least one copolymer in a polar aprotic solvent;
   C.II. adding a surfactant to form an organic phase;
   C.III. dispensing the organic phase into an aqueous mixture comprising at least one hydrophilic polymer and at least one hydrophilic active compound selected from CV-N to form a nanoemulsion; and
   C.IV. stabilising the nanoemulsion by stirring, and
(ii) producing an aqueous gel-like moisturizing composition comprising the steps of:
   D.I. mixing xanthan gum into glycerol by stirring until completely dispersed in the glycerol; and
   D.II. adding water and a preservative including citric acid, and stirring to form the aqueous gel-like moisturizing composition; and
(iii) mixing the stabilised nanoemulsion of step (i) to the aqueous gel-like moisturizing composition of step (ii) to form the alcohol-free, antimicrobial, moisturising hand sanitizer.

In one possible embodiment of the invention, the process for producing an alcohol-free, antimicrobial, moisturising hand sanitizer, consists of:
(i) producing a polymer-based nanoemulsion consisting of the steps of:
   A.I. co-dissolving at least one hydrophobic active compound selected from cannabidiol (CBD) and palmitic acid (PA), and at least one copolymer in a polar aprotic solvent;
   A.II. adding a surfactant to form an organic phase;
   A.III. dispensing the organic phase into an aqueous mixture comprising at least one hydrophilic polymer to form a nanoemulsion; and
   A.IV. stabilising the nanoemulsion by stirring; or
   B.I. dissolving at least one copolymer in a polar aprotic solvent;
   B.II. adding a surfactant to form an organic phase;
   B.III. dispensing the organic phase into an aqueous mixture comprising at least one hydrophilic polymer and at least one hydrophilic active compound selected from cyanovirin-N (CV-N) to form a nanoemulsion; and
   B.IV. stabilising the nanoemulsion by stirring, or
   C.I. co-dissolving at least one hydrophobic active compound selected from CBD and PA, and at least one copolymer in a polar aprotic solvent;
   C.II. adding a surfactant to form an organic phase;
   C.III. dispensing the organic phase into an aqueous mixture comprising at least one hydrophilic polymer and at least one hydrophilic active compound selected from CV-N to form a nanoemulsion; and
   C.IV. stabilising the nanoemulsion by stirring; and
(ii) producing an aqueous gel-like moisturizing composition consisting of the steps of:
   D.I. mixing xanthan gum into glycerol by stirring until completely dispersed in the glycerol; and
   D.II. adding water and a preservative selected from citric acid, and stirring to form the aqueous gel-like moisturizing composition; and
(iii) mixing the stabilised nanoemulsion of step (i) to the aqueous gel-like moisturizing composition of step (ii) to form the alcohol-free, antimicrobial, moisturising hand sanitizer.

The at least one copolymer may be poly (lactic-co-glycolic acid) or PLGA, or alternatively, any similar biocompatible and biodegradable polymer, including polylactic acid, polyglycolic acid, or poly 8-caprolactone. Preferably the copolymer is PLGA.

The polar aprotic solvent may comprise of either ethanol or acetone or may be a blend of ethanol and acetone. Preferably the polar aprotic solvent is a co-solution of acetone/ethanol (3:1).

The surfactant may comprise any surfactant having a Hydrophile-Lipophile Balance (HLB) value of greater than 10. Preferably, the surfactant is polysorbate 80, also known as Tween 80®.

The at least one hydrophilic polymer may be any one or more of polyvinyl alcohol (PVA) and polyethylene glycol (PEG), for example, PEG 4000. Preferably, the hydrophilic polymer is hydrolysed PVA.

The phosphate buffer (or PBS) may comprise a pH of from about 7.2 to about 7.6, more preferably, about 7.4 at 0° C.

In one embodiment of the invention, the aqueous gel-like moisturizing composition may comprise or consist of a mixture of xanthan gum—0.0007 kg: glycerol—0.067 L: citric acid—0.005 kg: water—1 L, including a ±5% variance of each amount.

In one embodiment of the invention, the polymer-based nanoemulsion functionalized with CV-N may comprise or consist of a mixture of 0.1-1 mg CV-N, 5-25 mg PLGA, 1-2 mL acetone/ethanol (3:1), 0.1-0.25 mL polysorbate 80 surfactant, 2-4 mL PBS, and 6-8 mL polyvinyl alcohol (1% w/v), including any possible subrange or amount within these ranges.

In another embodiment of the invention, the polymer-based nanoemulsion functionalized with CBD and/or PA may comprise or consist of a mixture of 0.5-2 mg CBD and/or 0.1-1 mg PA, 5-25 mg PLGA, 1-2 mL acetone/ethanol (3:1), 0.1 -0.25 mL polysorbate 80 surfactant, 2-4 mL PBS, and 6-8 mL polyvinyl alcohol (1% w/v), including any possible subrange or amount within these ranges.

Furthermore, in one particular embodiment of the invention, the polymer-based nanoemulsion is mixed with the aqueous gel-like moisturizing composition at a ratio of about 1:99 (i.e., 1-10:90-99) to form the alcohol-free, antimicrobial, moisturising hand sanitizer.

According to a second aspect of the invention, there is provided an alcohol-free, antimicrobial, moisturising hand sanitizer produced according to the method of the invention.

For example, the hand sanitizer may contain an active compound selected from any one of (a) CV-N, CBD or PA; (b) both of CV-N and CBD, or both of CBD and PA, or both of CV-N and PA, or (c) all three of CV-N, CBD and PA.

The hand sanitizer may be an antimicrobial agent, including any one or more of an antibacterial, antiviral or antifungal agent.

The polymer-based nanoemulsion may comprise:
i. an inner nanoemulsion matrix comprised of a polar aprotic solvent, at least one copolymer and a surfactant;
ii. an outer shell comprising one or more hydrophilic polymers and a buffer; and
iii. one or more of the active compounds,
   wherein where active compound(s) is(are) hydrophobic, the active compound(s) is(are) comprised in the inner nanoemulsion matrix, and wherein the active compound(s) is(are) hydrophilic, the active compound(s) is(are) comprised in the outer shell.

The polymer-based nanoemulsion may consist of:
A.a) an inner nanoemulsion matrix consisting of a polar aprotic solvent, at least one copolymer, a surfactant and any one or more active compound(s) selected from CBD and PA; and
A.b) an outer shell consisting of one or more hydrophilic polymers and a buffer, or
B.a) an inner nanoemulsion matrix consisting of a polar aprotic solvent, at least one copolymer, and a surfactant; and
B.b) an outer shell consisting of one or more hydrophilic polymers, a buffer and an active compound selected from CV-N, or
C.a) an inner nanoemulsion matrix consisting of a polar aprotic solvent, at least one copolymer, a surfactant and any one or more active compound(s) selected from CBD and PA; and
C.b) an outer shell consisting of one or more hydrophilic polymers, a buffer and an active compound selected from CV-N.

The outer shell may, in particular, comprise an aqueous solution of hydrophilic polymers such as any one or more of polyvinyl alcohol (PVA) and polyethylene glycol (PEG), for example, PEG 4000. Preferably, the hydrophilic polymer is hydrolysed PVA.

The at least one copolymer may be poly (lactic-co-glycolic acid) or PLGA, or alternatively, any similar biocompatible and biodegradable polymer, including polylactic acid, polyglycolic acid, or poly 8-caprolactone. Preferably the copolymer is PLGA.

The polar aprotic solvent may comprise of either ethanol or acetone, or may be a blend of ethanol and acetone. Preferably the polar aprotic solvent is a co-solution of acetone/ethanol (3:1).

The surfactant may comprise any surfactant having a Hydrophile-Lipophile Balance (HLB) value of greater than 10. Preferably, the surfactant is polysorbate 80, also known as Tween 80®.

Typically, the buffer is phosphate buffered saline (PBS).

The xanthan gum may be dispersed in the glycerol.

According to a further aspect of the invention, there is provided a method for sanitising hands and optionally surfaces with the alcohol-free, antimicrobial, moisturising hand sanitizer according to the invention.

According to a further aspect of the invention, there is provided a method for inhibition of microbes, including bacteria, viruses and fungi, in particular viruses with the alcohol-free, antimicrobial, moisturising hand sanitizer according to the invention.

According to a further aspect of the invention, there is provided a use of the antimicrobial, moisturising hand sanitizer according to the invention in a method of sanitising hands and/or inhibition of microbes, including bacteria, viruses and fungi, in particular viruses.

The viruses may be any one or more of HIV-1 or coronaviruses including SARS-CoV-2 or MERS-CoV.

### BRIEF DESCRIPTION OF THE DRAWINGS

The invention will now be further described, by way of example, with reference to the accompanying drawings.

FIG. 1 shows a graphical representation of the alternative nanoemulsion delivery system solutions, incorporating any one or more of the active compounds CV-N, CBD and PA, including their sites of incorporation in the nanoemulsion delivery system;

FIG. 2 shows droplet size and size distributions of the control nanoemulsion (i.e. without active compound added;

FIG. 3 shows BSA (A) and CV-N nanoemulsion (B) sizes and distributions in water;

FIG. 4 shows BSA solution treated with the CV-N nanoemulsion;

FIG. 5 shows CBD nanoemulsion size distributions;

FIG. 6 shows antiviral activity of the CBD nanoemulsion sanitizer against SARS-CoV-2 pseudovirus;

FIG. 7 shows droplet size and size distributions of the PA nanoemulsion;

FIG. 8 shows the skin irritancy visual score results;

FIG. 9 shows skin irritancy chromameter erythema results;

FIG. 10 shows skin irritancy maximum irritancy values;

FIG. 11 shows the concept design for production of nanoemulsion hand sanitizers; and

FIG. 12 shows the cytotoxicity assay in a 96 well plate.

### DETAILED DESCRIPTION OF THE INVENTION

The current invention relates to a method for producing an alcohol-free, antimicrobial, moisturising hand sanitizer comprising (i) a polymer-based nanoemulsion including one or more active compounds selected from cyanovirin-N (CV-N), cannabidiol (CBD) and palmitic acid (PA); and (ii) an aqueous gel-like moisturizing composition comprising xanthan gum, glycerol and a preservative such as citric acid. The invention further relates to an alcohol-free, antimicrobial, moisturising hand sanitizer produced by the method of the invention, as well as to use of the hand sanitizer as an antimicrobial, in particular an antiviral agent.

The following description of the invention is provided as an enabling teaching of the invention, is illustrative of the principles of the invention and is not intended to limit the scope of the invention. It will be understood that changes can be made to the embodiment/s depicted and described, while still attaining beneficial results of the present invention. Furthermore, it will be understood that some benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention.

The applicants have developed a novel alcohol-free hand sanitizer technology as an alternative to alcohol-based hand sanitizers (ABHS), comprising of safe and naturally occurring antimicrobial compounds embedded within a viscous polymer-based nanoemulsion system with a moisturizing effect on hands. The active antimicrobial compounds of the present technology include any one or more of:
i) cyanovirin-N (CV-N), a naturally occurring lectin (protein) with broad-spectrum antiviral activity;
ii) cannabidiol (CBD), a phytocannabinoid molecule with multifunctional applications and numerous health benefits extracted from the cannabis plant, and
iii) palmitic acid (PA), a saturated fatty acid known to be a potent antimicrobial agent.

Aspects of the present invention, therefore, entail an alcohol-free, nanoemulsion-based antimicrobial, moisturising hand sanitizer comprising:
a) a delivery system containing one or more active compounds selected from CV-N, CBD, and PA; and
b) an aqueous gel-like moisturizing composition comprising of xanthan gum and glycerol together with citric acid as a preservative.

A further aspect of the present invention relates to a manufacturing process for production of the alcohol-free, nanoemulsion-based antimicrobial, moisturising hand sanitizer.

Each of the active compounds used in the present invention exhibits different features in relation to speed, persistence, and spectrum of activity against viruses, pathogens and bacteria. Accordingly, these combined features provides the present invention with unique properties for improved and multifunctional antimicrobial activity against harmful bacteria, viruses and other pathogens.

Specifically, the active compounds CV-N, CBD and PA have been chosen for inclusion in the present invention for their features as set out below:
CV-N: Provides a broad antiviral activity spectrum for enveloped viruses including HIV and SARS-CoV-2 inhibition
CBD: Provides an extra protective layer against resistant bacteria and other harmful pathogens
PA: Acts as an active supplement required to maintain and restore the skin's natural barrier functionality against drug-resistant and harmful pathogens.

In addition, the polymer based nanoemulsion antimicrobial agent's delivery system and natural gum-based gel topical dosage formulation provide a balance of antimicrobial efficiency, while simultaneously improving the condition of the user's skin over long-term use, and minimising dry time to avoid unpleasant feel on the user's hands associated with prolonged skin surface “wetness”.

Lectins are a cluster of proteins that are naturally produced mainly in plants, a few dairy products, and eggs. Their structural compositions are carbohydrate-rich and have high binding specificity to sugar groups on either cellular or unicellular organisms and cause agglutination. These properties are desirable for antimicrobial activity and Mitchell et al. (2007) showed that the sugar affinity towards glycoproteins of enveloped viruses was similar and resulted in the virus collapse.

Cyanovirin-N (CV-N) has also been shown to have these properties towards enveloped viruses with class 1 fusion proteins, such as the gp120/gp41 fusion glycoproteins of the Human Immunodeficiency Virus (HIV) and inhibits cellular infection by the HIV virus. The broadspectrum antiviral activity of CV-N is likely to also inhibit viral infections by SARS-CoV-2 as this is also an enveloped virus with class 1 fusion proteins.

Cannabidiol (CBD) is gaining attraction exponentially due to its numerous health benefits through its ability to induce and regulate various cellular functions beneficial to the body. Recent studies have also shown CBD to have potent antimicrobial activity against gram positive bacteria (Wassmann et al., 2020; Martinenghi et al., 2020). CBD has been included by the applicant in the present invention as a helper compound against resistant bacteria and to serve as an extra protective layer against other pathogens.

Palmitic acid (PA) is a natural saturated fatty acid and a precursor for numerous endogenous and exogenous phospholipids. It is a precursor for ceramides forming the skin lipid barrier and widely used in personal care products as a surfactant or emulsifier. A number of saturated fatty acids including PA have been shown to have antibacterial activity against a broad range of gram-positive bacteria and some fungi (Nguyen et al., 2016).

Xanthan gum has gained much interest as a good gelling agent for the preparation of aqueous gels with a pleasant and cool skin feeling on application. Xanthan gum has the ability to form high viscous solutions at low concentrations and has been shown to be stable at various temperatures and wide pH range. Moreover, it is compatible with all the ingredients such as salt, metallic cations, surfactants, or bioactive compounds that are present in skin-care products. Owing to such excellent properties, xanthan gum has found many applications in oral and topical formulations, cosmetics and food as suspending or stabilising agent, thickening, emulsifying, film forming, gelling and release control agent in hydrophilic matrix formulations (Abdulsalam et al. 2018, Pawan et al., 2004).

The applicant's technology presents a far greater opportunity beyond the current COVID-19 pandemic. It presents a quick, noncomplex, and cost-effective process methodology for producing safe and effective alcohol-free hand sanitizers. It employs the use of naturally occurring antimicrobial actives with broad-spectrum activity and poses none of the safety and health hazards associated with ABHS. It provides a dermatologically safe, moisturizing product suitable for repeated use while maintaining the skin's microflora and a surplus precursor lipid for the epidermal (i.e. ceramide) and sebaceous (i.e. triglycerides) skin lipids. Furthermore, it is an alternative for anti-alcohol users, whether individual due to specific phobias, or groups/populations due to religious beliefs.

The exemplary examples below are provided as enabling teachings of the invention, illustrative of the principles of the invention and are not intended to limit the scope of the invention. It will be understood that changes can be made to the embodiments depicted and described, while still attaining beneficial results of the present invention.

Furthermore, it will be understood that some benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention.

### EXAMPLES

### 1. Aims and Objectives

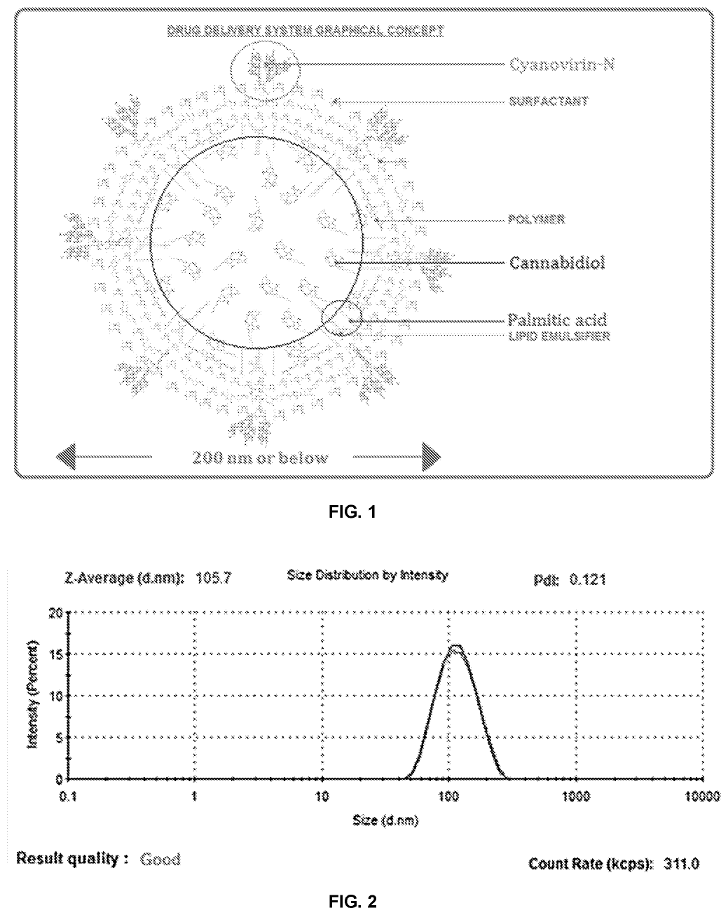

The primary aim of the study was to develop safe and stable aqueous delivery systems incorporating any one or more of Cyanovirin-N (CV-N), Cannabidiol (CBD) and Palmitic acid (PA) as active compounds. FIG. 1 shows a graphical representation of the alternative nanoemulsion delivery system solutions, incorporating any one or more of the active compounds CV-N, CBD and PA, including their sites of incorporation in the nanoemulsion delivery system. For example, the nanoemulsion delivery system may comprise only CV-N, CBD, or PA, or all of CV-N, CBD and PA, or only CV-N and CBD, only CBD and PA, or only CV-N and PA. These nanoemulsion delivery systems were then be infused with a viscosity builder solution of Xanthan gum and glycerol in order to produce safe and cheap lubricating gel-based hand sanitizers.

1.1. Specific objectives

The specific objectives include:
Synthesis of nanoemulsion incorporating CV-N
Synthesis of nanoemulsion incorporating CBD
Synthesis of nanoemulsion incorporating PA
Synthesis of Xanthan gum gel solution
Synthesis of the nanoemulsion sanitizers (nanoemulsion infusion into gel solution)
Physicochemical characterization and stability studies
Antiviral activity of nanoemulsion sanitizers against pseudoviruses in-vitro
In-vitro cytotoxicity studies
Skin irritancy patch tests

### 2. Materials and Methods

### 2.1. Materials for Nanoemulsion Synthesis

Cannabidiol was supplied by Tautomer (Pty) Ltd and purchased from REGENT Pharmaceuticals, UK. Cyanovirin-N was supplied by the NextGen Health cluster of the CSIR and Palmitic acid was purchased from DB Fine Chemicals, South Africa. Solvents were purchased from Sigma and include ethanol, acetone, acetonitrile, dimethyl sulfoxide (DMSO), ethyl acetate, dichloromethane (DCM) and oleic acid. Polyvinyl alcohol (PVA) (87-89 hydrolysed/Mw=13000-23000), stearic acid and phosphate buffer saline (PBS—pH 7.4) reagents were also obtained from Sigma Aldrich, South Africa.

### 2.2. Physicochemical Analysis Equipment

The Malvern Zetasizer nano series ZS (DLS) was used to determine the hydrodynamic size, size distributions and stability. Acsen pH meter from Lasec was used to monitor the pH of the emulsions and a Shimadzu SIL-20AXR/20ACRXR prominence High Pressure Liquid Chromatography (HPLC) for qualitative analysis (to check actives integrity post-formulation and during shelf-life).

### 2.3. Cell Culture Materials

The human lung cancer cells, A549 were a gift from the Scholefield lab (CSIR). Cells were cultured in Dulbecco's Modified Eagle's Medium (DMEM), containing L-glutamine and sodium pyruvate obtained from Life Technologies, NY (USA). Culture media was supplemented with 10% fetal calf serum (FCS) and Trypsin-EDTA 0.25% solution was used to detach adherent cells, both were purchased from Thermo Fischer Scientific, Johannesburg (South Africa). The MTT colorimetric cell proliferation assay was used to investigate toxicity and purchased from Sigma (Roche, Mannheim, Germany).

### 2.4. Methods

### 2.4.1. Synthesis of CV-N Nanoemulsion

Briefly, the nanoemulsion system functionalized with CV-N was prepared as follows: CV-N stock solution was prepared by dissolving 0.1 mg in 1 mL of PBS (pH 7.4) solution. The organic phase (internal) was prepared by dissolving PLGA in 2 mL of a co-solution of acetone/ethanol (3:1) followed by the addition of 10 to 20 μl (2 drops) of the Tween 80 surfactant. The polar phase (continuous) was prepared through mixing 4.5 mL of the buffering solution of PBS (pH 7.4) with 13.5 mL of a hydrolysed hydrophilic polymer, polyvinyl alcohol (1% w/v PVA). CV-N stock solution (100 μL) was added to the continuous phase while stirring (400 rpm) at room temperature (±21° C.).

The nanoemulsion was synthesised by adding the organic phase into the continuous phase while stirring and the spontaneous precipitation reaction resulted in the self-assembly of a stable nanoemulsion via nucleation. The nanoemulsion was then stirred under a fume hood for 2 hours to evaporate the solvents. A control nanoemulsion without the addition of lectins was also prepared following the exact method of synthesis as described above.

### 2.4.2. Synthesis of CBD Nanoemulsion

The organic phase was prepared by co-dissolving PLGA and CBD (10 mg each) in a 2 mL co-solution of acetone/ethanol (3:1) followed by the addition of 10 to 20 μl (2 drops) of Tween 80. The polar phase was prepared by mixing 4.5 mL PBS solution (pH 7.4) and 13.5 mL polyvinyl alcohol (1% w/v PVA). To form the nanoemulsion, the organic phase was added into the continuous phase while stirring. The spontaneous precipitation resulted in a stable nanoemulsion encapsulating CBD and was stirred under a fume hood for 2 hours to evaporate the solvents.

### 2.4.3. Synthesis of PA Nanoemulsion

The organic phase was prepared by dissolving 10 mg of PLGA and 1 mg of PA in a 2 mL co-solution of acetone/ethanol (3:1) followed by the addition of 10 to 20 μl (2 drops) of Tween 80. The continuous polar phase was prepared by mixing 4.5 mL of PBS pH 7.4 and a hydrophilic polymer, polyvinyl alcohol (13.5 mL). To form the nanoemulsion, the organic phase was added to the continuous phase while stirring and the spontaneous precipitation resulted in the self-assembly of PLGA/PA nanodroplets. The system was then stirred under a fume hood for 2 hours to evaporate the solvents.

### 2.4.4. Synthesis of Xanthan Gum/Glycerol Solution

Due to the hygroscopic nature of various gums and their tendency to hydrate upon immediate contact with water, they often result in the formation of gum balls or lumps of dry powder where the water couldn't penetrate. To overcome this, glycerol was used to disperse the gum powder and minimize hydration before gel formation. Therefore, in a typical procedure, varying xanthan gum amounts from 0.025 g to 0.3 g were accurately weighed into 250 mL conical flask containing 10 mL of Glycerol. The mixture was magnetically stirred slowly to allow complete xanthan gum dispersion. To the flask, 150 mL of deionised water at 60° C. was added while stirring vigorously at 500 rpm to remove air bubbles and gum balls during the gel formation. The gel was kept under vigorous stirring for 30 min and the final volume of the gel ranged between 155-160 mL. The compositions of the prepared gels are summarized Table 1.

TABLE 1
Formulation compositions for gel synthesis

| Gel | Xanthan gum (g) | Water (ml) | Glycerol (ml) |
|---|---|---|---|
| X1 | 0.025 | 150 | 10 |
| X2 | 0.05 | 150 | 10 |
| X3 | 0.075 | 150 | 10 |
| X4 | 0.1 | 150 | 10 |
| X5 | 0.2 | 150 | 10 |
| X6 | 0.3 | 150 | 10 |

### 2.4.5. Synthesis of Sanitisers

Final concentrations of 10% (v/v) of the nanoemulsions were infused into the gel to form all the sanitizers. Each sanitizer was prepared by adding 1 mL of the nanoemulsion into 9 mL of the gel solution while stirring moderately at room temperature.

### 2.4.6. Hydrodynamic Size and Size Distribution

The hydrodynamic size, size distributions and stability of the nanoemulsion were determined by a Dynamic Light Scattering (DLS) technique using the Malvern Zetasizer Nano ZS. The DLS instrument measures the Brownian motion, the random movement (fluctuation) of submicron particles in a solution to determine the hydrodynamic size.

Briefly, a laser beam is used to illuminate the sample solution, the incident laser beam gets scattered in all direction and the intensity measured by a detector. Stability of submicron particles can also be determined by DLS over time through continuous sample analysis. Samples for analysis were prepared in deionized water, diluted 300 to 400 times and a disposable zetasizer cuvette was used for the analysis.

### 2.4.7. Stability

The stability of emulsions is by far the most crucial property to consider and achieve as most failures occur during this phase. It is the ability of a nanoemulsion to maintain its integral physicochemical properties (e.g. size and distribution) for long durations and unaffected by most environmental changes (i.e. temperature). To achieve good stability, key considerations such as surfactant properties and solvent thermodynamics are of paramount importance.

Degradation of emulsions (instability) can be through phase separation (1), Ostwald ripening (2), aggregation (3) or phase inversion (4). Instabilities can be observed through precipitations or sedimentations by the naked eye although some are slow and take longer.

In the study we investigated the stability of the emulsions and the sanitizers through continual analysis of the size and size distributions of both systems. A Malvern Zetasizer Nano Series ZS was used and samples were prepared by adding 100 μL of the samples into 5 mL of deionised water.

### 2.4.8. In-Vitro Inhibition Assays

The inhibition activity of CV-N, CBD, PA and the combinations thereof incorporated in the nanoemulsion delivery systems were tested in a TZM-bl neutralisation assay. The TZM-bl neutralisation assay mimics the inhibition of free viral particles infection of cells. Briefly, the TZM-bl neutralization assay was performed by preparing a dilution series of the inhibitors in 100 μL of the growth medium (DMEM) with 10% Fetal Bovine Serum (FBS) in a 96-well plate in duplicate. This was followed by the addition of 100 TCID₅₀ of pseudovirus in 50 μL of growth medium and incubated for one hour at 37° C. Then 100 μL of TZM-bl cells at a concentration of 1×10⁵ cells/mL containing 37.5 μg/mL of DEAE-dextran will be added to each well and cultured at 37° C. for 48 h. Infection will be evaluated by measuring the activity of the firefly luciferase.

Titres were calculated as the inhibitory dilution that causes 50% reduction (ID₅₀) of relative light unit (RLU) compared to the virus control (wells with no inhibitor) after the subtraction of the background (wells without both the virus and the inhibitor). The luciferase assay was performed with the Bright-Gloluciferase assay kit (Promega, USA) according to the manufacturer's instructions and luciferase activity has been expressed in terms of relative luciferase units (RLUs).

### 2.4.9. In-Vitro Cytotoxicity Assays

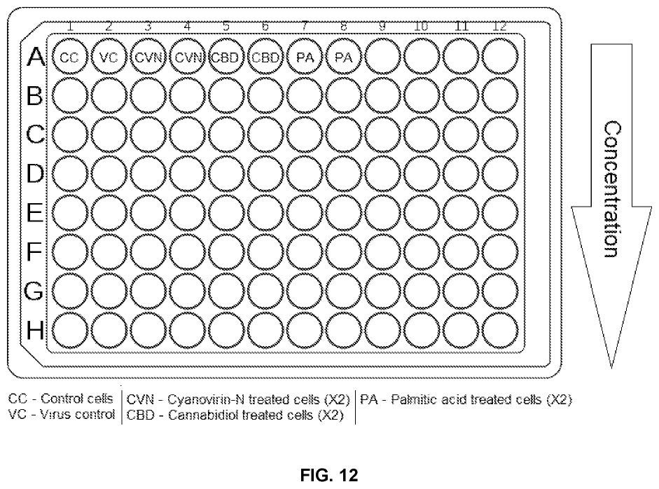

The cells were grown and maintained at a confluency of 0.5-5×10⁶ cells/ml, in DMEM/F12 penicillin (50 μg/ml), streptomycin (50 μg/ml) and Neomycin (100 μg/ml) and 10% heat inactivated foetal calf serum (FCS). The cells were incubated at 37° C. in a 5% CO₂ (g) humidified incubator and passaged 2-3 times a week. Cell viability was determined by making use of the trypan blue exclusion test. Cells were seeded in 96 well plates at densities of 5×10⁴ cells/ml using 100 μl per well and incubated for 48 hours before treatment. Cells were incubated with different concentrations of the various microemulsions at 100 μl as indicated in FIG. 12. All test samples were diluted in complete media without antibiotics. The control samples: Cannabidiol and Palmitic acid were solubilised in DMSO before diluted in culture media (max 2% v/v). Cyanovirin-N was prepared in PBS (pH 7.4) and further diluted in culture media without DMSO.

The MTT assay was performed according to the manufacturer's instructions. In summary, this non-radioactive, colorimetric assay system using MTT was first described by Mosmann, T (1983) and has been improved in subsequent years by several other investigators. The MTT assay is used to measure cellular metabolic activity as an indicator of cell viability, proliferation and cytotoxicity. This colorimetric assay is based on the reduction of a yellow tetrazolium salt (3-(4,5-dimethylthiazol-2-yl)-2,5-diphenyltetrazolium bromide or MTT) to purple formazan crystals by metabolically active cells. The viable cells contain NAD(P)H-dependent oxidoreductase enzymes which reduce the MTT to formazan. The insoluble formazan crystals are dissolved using a solubilisation solution and the resulting coloured solution is quantified by measuring absorbance at 500-600 nanometers using a multi-well spectrophotometer. After incubation of the test compounds for 24 hours, MTT, dissolved in PBS pH 7.4 (100 μg/well), was added to the cells and incubated for 4 hours at 37° C. The untransformed MTT was removed by careful aspiration and the formazan crystals formed, were dissolved in 100 μl of the solubilising solution. The absorbance was read at 550 nm with a reference filter of 650 nm. Percentage viability was calculated by the absorbance intensity of the cells incubated with the microemulsions as a function of that of the untreated cells. Three independent experiments were performed and each compound tested in in triplicate wells.

### 2.4.10. Skin Irritancy

The objective of the study was to determine skin irritancy potential of the nanoemulsion hand sanitizer products of the invention. The study was conducted according to internationally recognized Good Clinical Practice Guidelines. Permission to conduct the study was covered under the protocol approval for project MREC/H/48/2014: CR of the Research, Ethics and Publications Committee of the Sefako Makgatho Health Sciences University. Recruitment for the study began two weeks before the commencement of the study and approximately 25 panellists were recruited according to the study requirements, to commence the study with a test panel of 20. Potential panellists attended a series of study briefings held during the week prior to the study. Panellists were verbally briefed and given the Study Instructions, Informed Consent forms (plus coloured duplicate copies of the latter).

Panellists were given specifically timed appointments for the whole study. Unless otherwise stated in the summary, twenty adult female volunteers were used, of whom five had sensitive skins (as determined from the subject enrolment questionnaire). Signed consent forms were obtained from all subjects. Subjects with a history of clinical dermal abnormalities or who are taking or using medication which may affect test results through mediation of the inflammatory response were excluded. Twenty adult female volunteers were used, of whom five had sensitive skin. Signed consent forms were obtained from all subjects.

Briefly, the study procedure was as follows; Controls and products were applied to the inner forearm of each volunteer in a randomized, blinded pattern at zero hours and repeated on the same position at 24 hours. A measured volume of control or pieces of product was placed in the respective chamber and the chamber attached to the forearm. Products were tested by using specially designed aluminium Finn Chambers on Scanpore® tape for occluded sites or modified Hilltop chambers for unoccluded sites. The patches were observed at 0, 24, 48, 72 and 96 hours after application. Patch areas were covered with the chambers for the first 2×23 hours, thereafter the chambers were removed.

The protocol uses 1% sodium lauryl sulphate (SLS) solution as a positive control and de-ionized water as a negative control. A measured amount of 0.02 ml of sodium lauryl sulphate and de-ionized water was applied. The following visual rating system was used to classify the reactions. Colour assessments were performed in a “double blind” manner in 2 ways: Visual score where; 0=no reaction, 1=slight erythema, 2=strong erythema confined to the immediate contact area and 3=strong erythema spreading beyond the contact area Irritancy levels are classified by our laboratory, based on the visual scores, as follows: product mean visual score+SD >1.5—Product is irritant product mean visual score+SD<=1.5 and >negative control—Product is of low irritancy potential. Products mean visual score+SD<=negative control—Product is non-irritant. The second was through instrumental score using the Minolta Cr400 chromameter using the a* value, which measures colour on the red/green axis.

### 3. Results and Discussions

### 3.1. Nanoemulsion—Control System

### 3.1.1. Physicochemical Results Discussions

Development of the nanoemulsion system was successful through an oil-in-water (O/W) single nanoemulsion via rapid nanoprecipitation technique. The physical properties in terms of size (diameter, nm) and distributions of the nanoemulsion were determined by DLS analysis.

FIG. 2 shows a graphical representation of the control nanoemulsion properties and the average droplets sizes were 105.7 nm with homogeneous size distributions.

The pH and physical properties were used as parameters to investigate stability (Table 3). The initial pH of the control nanoemulsion was 5.71 (±0.03), stored at room temperatures (±21° C.) and the properties were unaltered over a 5 months period. The size reduction by 1.8 nm was expected (57%) and a result of evaporating solvents.

TABLE 3
Physicochemical properties of control nanoemulsion over 5 months

| Experiment | Date synthesized and | | Date analysed and | | |
|---|---|---|---|---|---|
| no | results | | results | | Difference (%) |
| PN34 | Feb. 4, 2020 | 105.7 nm/0.121 | Mar. 9, 2020 | 103.9 nm/0.126 | 2% |
| pH | | 5.71 | | 5.72 | |

### 3.2. Cyanovirin-N Sanitiser

### 3.2.1. Physicochemical Results Discussions

CV-N was successfully functionalized onto the surface droplets of the nanoemulsion system with appreciable average sizes of 155.4 nm and size distributions were just below acceptable ranges (PDI≤50.3). Size increase is the first parameter observed in determining success and results in Table 4 suggests successful functionalization. To motivate the use of size as a determining factor, we analysed the stock solution of CV-N and 60-70% of sizes ranged between 25-50 nm. However, qualitative analysis through a high-resolution transmission electron microscope (TEM) is required and recommended for confirmation. The stability was investigated and determined through similar method used for the control nanoemulsion and described above.

TABLE 4
Physicochemical properties of CVN nanoemulsion over 5 months

| Experiment | Date synthesized and | Date analysed and | Difference |
|---|---|---|---|
| no | results | results | (%) |
| PN42 | Feb. 4, 2020 | Mar. 9, 2020 | −17% |
| | 155.4 nm/0.265 | 129.3 nm/0.336 | |
| pH | 6.47 | 6.46 | |

These results show a higher size reduction by at least 17% and exceed the 7% threshold. The pH was within acceptable ranges after 5 months. The functionalization process mainly occurs through occlusion onto the outer surfactant layer formed by hydrophilic polymers. The occlusion process is a non-chemical reaction and also the droplets are semi-solids that are in continual movement and colliding due to Brownian motion. The consistent collision of droplets may result in 2 outcomes; first being the loosening followed by dislodging of some CVN from the droplets surfaces and/or the second being the protein further internalized (pushed in) as the droplets are semi-solids. Although, both outcome possibilities of the protein did not degrade nor affect its functionality, the protein remained intact in the nanoemulsion and continued to function as intended, the integrity was confirmed through HPLC analysis before and after synthesis.

Further to confirmation by HPLC analysis, we designed a study to validate our hypothesis and preliminary results support our proposed mechanisms for the observed size reductions. As stipulated in the project proposal, our strategy was to develop a delivery system with high binding affinity towards viral surface proteins. The nanoemulsion synthesis aimed and achieved to functionalize (attach) CV-N non-chemically through occlusion onto the droplets surface. In this study, we investigated the affinity of a protein towards binding to the surface of functionalized droplets within the nanoemulsion and we used size as a determining parameter.

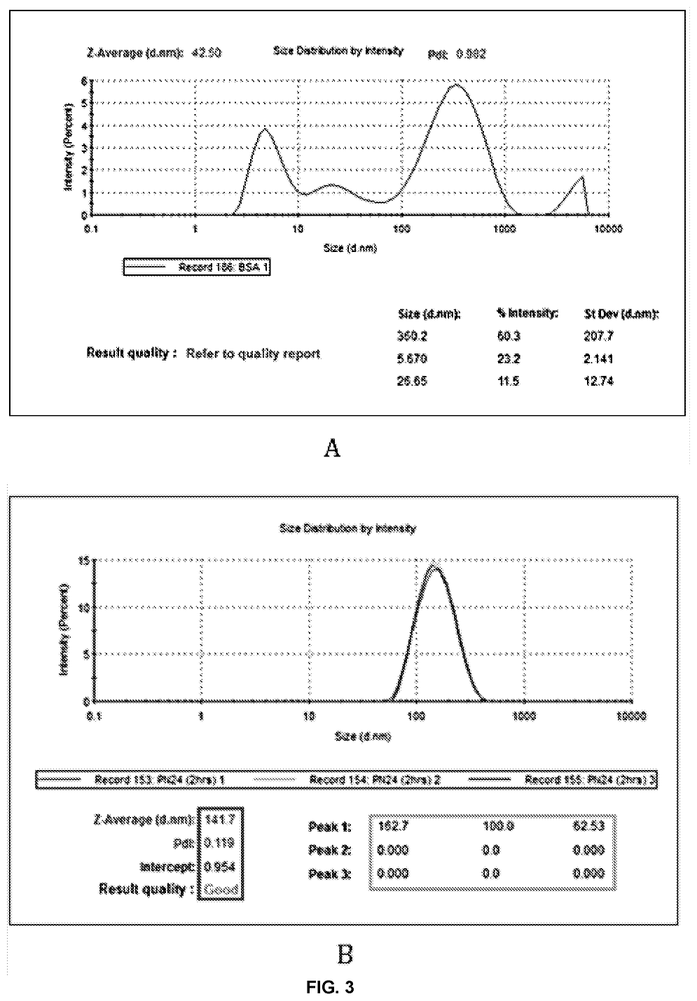

Proteins are long polypeptide chains synthesized from hydrophobic and hydrophilic amino acids; the folding arrangement is responsible for the solubility in water. The non-polar (hydrophobic) amino acids form the core (inside) and the polar (hydrophilic) form the outer surface resulting in quaternary proteins with a globular (round) three-dimensional shape. We employed the use of various concentration solutions of a 66.5 kDa protein; Bovine Serum Albumin (BSA) and when analysed through DLS, the sizes (diameter) of a single quaternary globular structure were around ±5 nm (FIG. 3A) and literature confirmed the results. The protein size distributions vary (5-360 nm) in water and is due to the lack of buffering ions as those found in the blood, thus the clustering of protein molecules to form agglomerates.

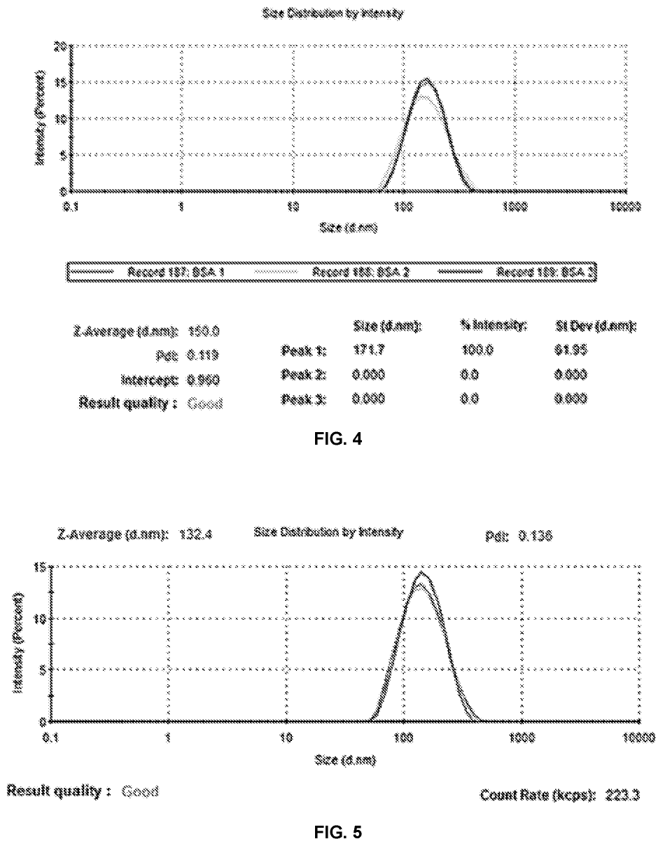

The BSA solution was then treated with a few drops (1 to 5 drops) of the CV-N nanoemulsion and analysed immediately. The initial sizes of the droplets in the nanoemulsion were around 141.7 nm with homogeneous size distributions and can be seen in FIG. 3B above. The sizes and size distributions of the treated BSA solution were rapidly and significantly improved with droplet sizes increasing by at least ±8 nm. FIG. 4 below shows the increase in sizes and the homogenous size distributions in just a few seconds. Moreover, the study has been performed in duplicate and the results were found to be the same.

3.2.2. HIV-1 Pseudovirus In-Vitro Inhibition Assay The antiviral activity of the CV-N nano-emulsion sanitizer was demonstrated in TZM-bl neutralization assay using 100 TCID50 and successful inhibitions were observed. Table 5 shows the inhibition profile of the sanitizer from high to low concentrations of CV-N.

Interestingly, reported IC50s of CV-N with infection of 100 TCID50 range from 2 to 31 nM (22-340 μg), which means that in order to inhibit 50% of the virus, at least 22 μg of CVN should be in solution. Our initial CV-N concentration in the nanoemultion sanitizer was 0.5 μg/mL followed by serial dilutions in sample preparations, and 0.025 μg (50 μL of sanitizer) was the total CV-N concentration used in well A. The average virus control (VC) was used to calculate the inhibition percentages.

TABLE 5
HIV-1 pseudovirus loads, inhibitions and CV-N concentrations

| | | | CV-N treated wells | | CV-N | % |
|---|---|---|---|---|---|---|
| Well | CC | VC | (×2) | | (μg/assay) | inhibition |
| A | 372 | 41147 | 421 | 420 | 0.0025 | 99.04 |
| B | 395 | 44615 | 1536 | 1364 | 0.0008 | 96.68 |
| C | 463 | 38603 | 4130 | 2735 | 0.000278 | 92.15 |
| D | 377 | 41684 | 6264 | 4692 | 9.25 × 10⁻⁵ | 87.48 |
| E | 431 | 44145 | 10334 | 8184 | 3.08 × 10⁻⁵ | 78.83 |
| F | 414 | 47072 | 23485 | 18192 | 1.03 × 10⁻⁵ | 52.36 |
| G | 410 | 45505 | 36075 | 29552 | 3.43 × 10⁻⁶ | 24.99 |
| H | 330 | 47212 | 38442 | 33937 | 1.14 × 10⁻⁶ | 17.27 |

### 3.3. Cannabidiol Sanitiser

### 3.3.1. Physicochemical Results Discussions

We investigated different concentrations of CBD to be incorporated in the nanoemulsions and 3 nanoemulsions were synthesized with 2, 10 and 20 mg of CBD content. The hydrodynamic sizes and size distributions of nanoemulsion systems with different concentrations were 114.7 nm, 108 nm and 158.3 nm respectively (see FIG. 5).

It seems unusual that the lower concentrations (2 mg) have slightly bigger sizes than the mid concentrations (10 mg), however the results are appreciable. To elaborate, like molecules (hydrophobic-hydrophobic) tend to aggregate when introduced in unfavourable environments (polar) and concentrations determines their arrangements. The arrangements of lower concentrations are normally irregular as compared to slightly higher concentrations and this phenomenon can be explained through principle of the critical micellar concentrations (CMC). CBD is more hydrophobic and smaller when compared to the encapsulating polymer matrix; this then means that the CBD molecules will precipitate faster as compared to the polymer. As a result, CBD will form small aggregates that become enclosed by the polymer, followed by hydrophilic surfactant polymers wrapping themselves on the micelles.

Where there is a lack of structural support from within, this results in the formation of irregular shapes whereas a compact core will maintain its morphological integrity. Also, the same concentration of the polymer was used and the 1:1 ratio (polymer/drug) produced ideal properties for CBD in this nanoemulsion system. The highest CBD concentration that can be incorporated in the core of 10 mg of the polymer is 10 mg.

The pH and physical properties were also used as parameters to investigate stability (Table 6). The initial pH of the nanoemulsion was 6.50 (±0.03), stored at room temperatures (±210C) and the properties were appreciable over a 5 months period. The size increase by at least 5 nm was observed (˜5%); however, the size distributions improved suggesting good stability.

TABLE 6
Physicochemical properties of CBD emulsions over 5 months

| Experiment | | First | | Last | Difference |
|---|---|---|---|---|---|
| number | Date | analysis | Date | analysis | (%) |
| PN44 HIV-1 | Feb. 4, 2020 | 132.4 nm/0.136 | Mar. 9, 2020 | 137.5 nm/0.115 | 4% |
| assay | | | | | |
| pH | | 6.50 | | 6.47 | |
| PN221 SARS | Apr. 8, 2020 | — | Mar. 9, 2020 | 156.1 nm/0.130 | — |
| assay | | | | | |
| pH | | | | 6.09 | |

### 3.3.2. HIV-1 Pseudovirus In-Vitro Inhibition Assay

The antiviral activity of CBD was also demonstrated in TZM-bl neutralization assay and successful inhibition of the pseudovirus from infecting the cells was achieved and can be observed in Table 7.

Table 7 below shows the initial virus concentrations including the control (no virus), and the CBD concentrations used to achieve inhibition activity of the sanitizer. Initial CBD concentrations (A, B and C) showed more than 90% inhibitions (LD90) and the LD50 was achieved with CBD concentrations around 3.70 μg in well D.

TABLE 7
HIV-1 pseudovirus loads, inhibitions and CBD concentrations

| | | | CBD treated wells | | CBD | % |
|---|---|---|---|---|---|---|
| Well | CC | VC | (×2) | | (μg/assay) | inhibition |
| A | 372 | 41147 | 163 | 147 | 5 | 99.64 |
| B | 395 | 44615 | 609 | 479 | 1.667 | 98.75 |
| C | 463 | 38603 | 2521 | 2123 | 0.555 | 94.69 |
| D | 377 | 41684 | 4786 | 4663 | 0.185 | 89.20 |
| E | 431 | 44145 | 9194 | 10067 | 0.0617 | 77.98 |
| F | 414 | 47072 | 15643 | 16925 | 0.0205 | 62.77 |
| G | 410 | 45505 | 29218 | 27043 | 0.0068 | 35.69 |
| H | 330 | 47212 | 31334 | 33747 | 0.0023 | 25.62 |

### 3.3.3. SARS-CoV-2 and MERS—CoV Pseudovirus Inhibition Assay

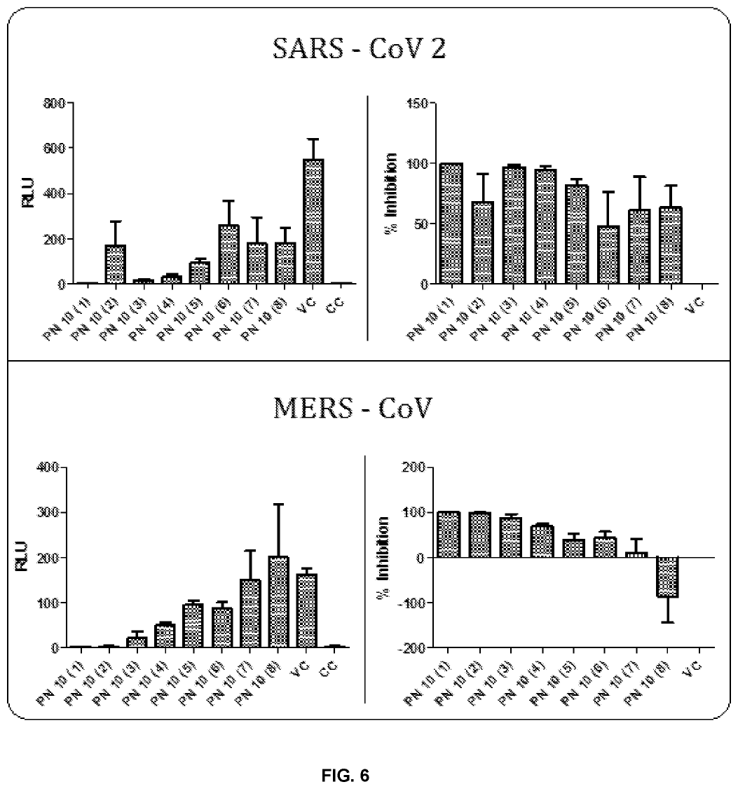

The antiviral activity of the CBD emulsions was demonstrated in a TZM-bl neutralization assay and successful inhibition of both SARS-CoV-2 and MERS-CoV pseudoviruses were achieved (FIG. 6).

### 3.4. Palmitic Acid Sanitiser

### 3.4.1. Physicochemical Results Discussions

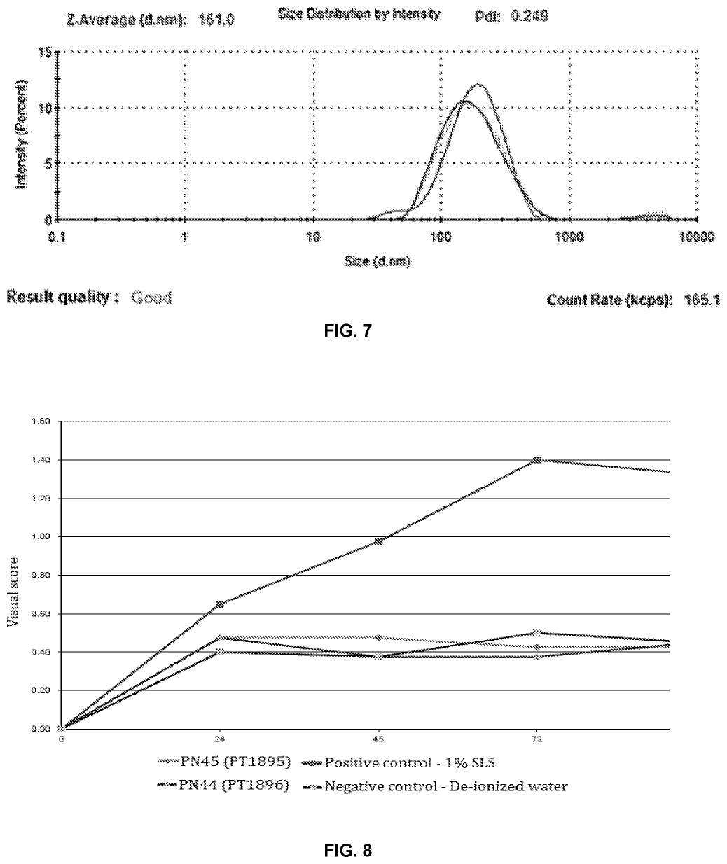

As can be seen below in FIG. 7, the nanoemulsion system incorporating the palmitic acid was developed successfully with desirable sizes and size distributions.

The pH and physical properties were also used as parameters to investigate stability (Table 8). The initial pH of the nanoemulsion was 6.54 (±0.03) followed by a slight increase; however, the size distributions improved suggesting good stability. The nanoemulsion was stored at room temperatures (±21° C.) and the properties were appreciable over a 5 months period. The size reduction by at least 33 nm was observed (˜21%) and although the stability improved, further investigations are necessary to determine the cause.

TABLE 8
Physicochemical properties of PA nanoemulsion over 5 months

| Experiment | | First | | | Difference |
|---|---|---|---|---|---|
| number | Date | analysis | Date | Last analysis | (%) |
| PN148 | Feb. 6, 2020 | 161 nm / | Mar. 9, 2020 | 127.6 nm / | −21% |
| | | 0.249 | | 0.147 | |
| pH | | 6.54 | | 6.64 | |

### 3.4.2. HIV-I Pseudovirus In-Vitro Inhibition Assay

The antiviral activity of the PA sanitizer was demonstrated using the TZM-bl neutralization assay and successful inhibition of the pseudovirus from infecting the cells was observed (Table 9).

TABLE 9
HIV-1 pseudovirus loads, inhibitions and PA concentrations

| | | | PA treated wells | | PA | % |
|---|---|---|---|---|---|---|
| Well | CC | VC | (×2) | | (μg/assay) | inhibition |
| A | 372 | 41147 | 759 | 758 | 0.25 | 98.26 |
| B | 395 | 44615 | 1575 | 1308 | 0.0833 | 96.70 |
| C | 463 | 38603 | 11784 | 7877 | 0.0277 | 77.53 |
| D | 377 | 41684 | 30272 | 28541 | 0.0092 | 32.78 |
| E | 431 | 44145 | 42575 | 37099 | 0.0031 | 8.94 |
| F | 414 | 47072 | 45954 | 44994 | 0.0010 | — |
| G | 410 | 45505 | 48365 | 46810 | 3.42 × 10-4 | — |
| H | 330 | 47212 | 38837 | 35165 | 1.14 × 10-4 | — |

### 4. In-Vitro Biological Assays

The cytotoxicity assays in A549 cells showed significant toxicity with CV-N and CBD controls (results not shown) and the MTT assay was used to measure cellular metabolic activity as an indicator of cell viability and proliferation.

The highest concentrations of the control nanoemulsion showed no toxicity with average cell survivals above 85%. Although the pure CBD showed significant toxicity, the CBD nanoemulsion showed no toxicity and significantly improved cell proliferations. CV-N nanoemulsions showed slight toxicity at higher concentrations.

However, the highest CV-N concentration used for the nanoemulsion sanitizer is 0.025 μg, therefore much lower compared to the lowest concentration (1.56 μg) in this toxicity study and is therefore considered to be safe.

### 5. Skin Irritancy

All panellists attended their daily visits, unless otherwise stated in the Summary. There were no adverse events during the study, unless listed in the Summary. In the case of adverse events, a report is submitted to Sefako Makgatho Health Sciences University Research and Ethics Committee. Data were collected at each visit as per the method described above. The product(s) was/were tested on the volar forearms of 20 panellists. The graphical representations of the results show the data for the product(s), the negative control (de-ionized water) and the positive controls (1% SLS).

### 5.1. Visual Score

The maximum irritancy (as calculated by the mean visual score+one standard deviation of the visual score) for the products and the controls is tabulated below. The irritancy limit for the visual score+one standard deviation (SD) is 1.5 on a 0-3 scale.

### 5.2. Chromameter

The change in Chroma a* values for each test site compared to baseline, is calculated as follows:

$$Delta {a}^{*} = ( Product {a}^{*} time t - Product {a}^{*} time 0 ) - ( untreated {a}^{*} time t - untreated {a}^{*} time 0 )$$

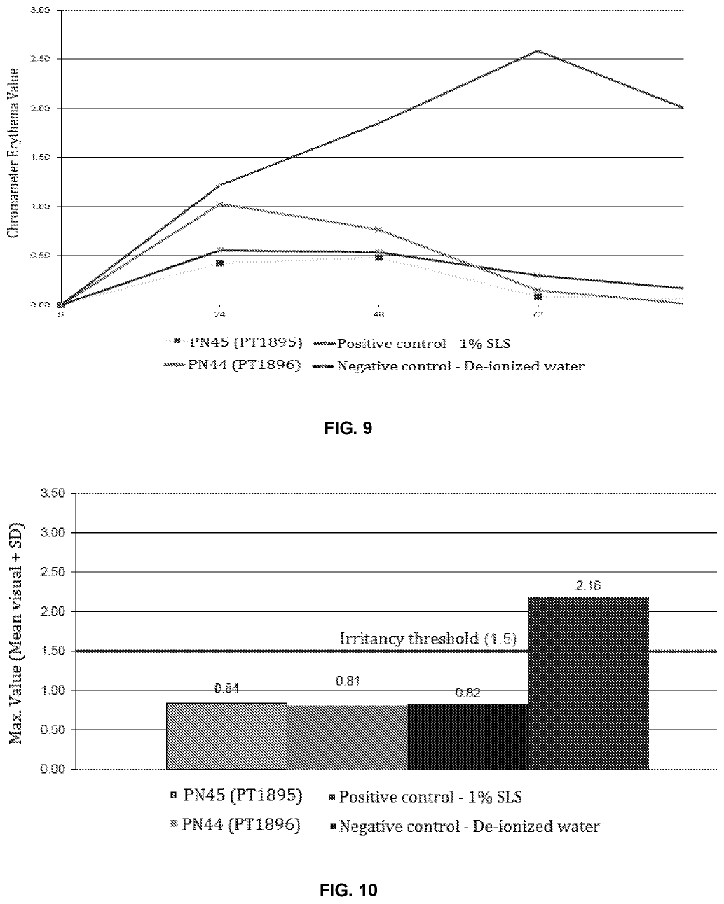

The delta a*values for all panellists for a given product at a given time point were averaged and plotted on the attached graphs. Chromameter a*value data patterns supported the visual erythemal assessment scores (provided in FIG. 9).

TABLE 10
Skin irritancy results

| | | Maximum | | Patch | | |
|---|---|---|---|---|---|---|
| | | value | | occluded | Neat or | |
| | | (mean | | (o) or | intact (n) | |
| | | visual | Time | Non- | or | Conclusion |
| | Code/ | score + | point | occluded | Diluted | (irritancy |
| Product | Description | sd) | (hours) | (n) | (d) | category) |
| Sample | PN45/PT1895 | 0.84 | 24-48 | O | N | Non-irritant |
| A | | | | | | |
| Hand | | | | | | |
| sanitiser | | | | | | |
| Sample I | PN44/PT1896 | 0.81 | 24 | O | N | Non-irritant |
| Hand | | | | | | |
| sanitiser | | | | | | |
| Sample | De-ionised | 0.82 | 72 | O | N | Non-irritant |
| C | water | | | | | |
| Negative | | | | | | |
| control | | | | | | |
| Sample | 1% Sodium | 2.18 | 72 | O | N | Irritant |
| H | Lauryl | | | | | |
| Positive | Sulphate | | | | | |
| control | | | | | | |

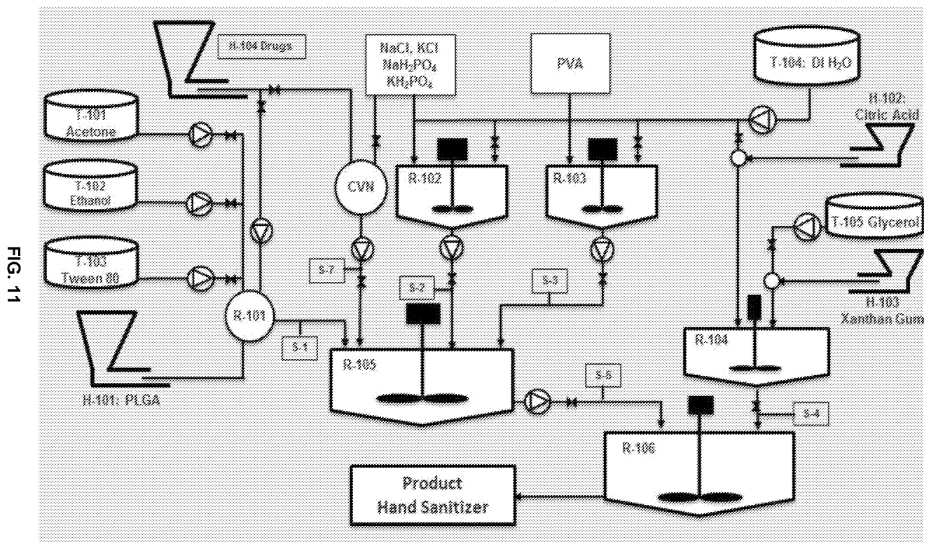

The study confirmed and concluded with no adverse events observed and FIG. 10 shows a graphical summary of the findings.

6. Method of production
6.1. Sanitiser preparation phases

The nano-emulsion hand sanitizer synthesis process described herein can be divided into 5 preparation phases including, 3 phases in the nanoemulsion preparation, gelation and lastly the fusion phase. The nanoemulsion phases include the preparation of the internal (P1) and the continuous (P2) phases followed by the nanoemulsion formation (P3).

### 6.2. Phase Preparations and Costing (1-3)

The organic phase preparations include 3 liquid streams for the solvent solution, one solid stream for the polymer and a reaction tank for solvation. An additional branched solid stream is required for active compounds (drugs), with a branch into solvation reaction tank (CBD and/or PA) and another into a protein reaction tank (CV-N). Table 11 below shows the required quantities of raw materials to produce 1 L of the solvent solution.

TABLE 11
Raw materials for the internal phase
STREAM 1: PLGA SOLUTION

| | | Required Qty/L (PLGA) | |
|---|---|---|---|
| Reagents | Unit | Liquid stream (L) | Solid Stream (kg) |
| Acetone | 1 L | 0.75 | 0 |
| (L) | | | |
| Ethanol (L) | 1 L | 0.25 | 0 |
| Tween 80 | 1 L | 0.025 | 0 |
| (L) | | | |
| PLGA (kg) | 1 kg | 0 | 0.1 |

The continuous phase comprises of a mixture solution (1:3) of PBS and PVA. Estimated quantities of raw materials needed to prepare 1 L for both the PBS solution (Table 12) and the PVA solution (Table 13) can be seen below.

TABLE 12
PBS raw materials
STREAM 2: PBS SOLUTION

Required Qty/L (PBS)

| Reagents | Unit | Liquid stream (L) | Solid Stream (kg) |
|---|---|---|---|
| NaCl (kg) | 1 kg | 0 | 0.008 |
| KCI (kg) | 1 kg | 0 | 0.0002 |
| NaH₂PO₄ | 1 kg | 0 | 0.00144 |
| (kg) | | | |
| KH₂PO₄ | 1 kg | 0 | 0.00024 |
| (kg) | | | |
| H₂O (L) | 1 | 1 | 0 |

TABLE 13
Estimated cost for PVA solution preparation
STREAM 3: 1% (W/V) PVA SOLUTION

Required Qty/L- PVA (1% w/v)

| Reagents | Unit | Liquid stream (L) | Solid Stream (kg) |
|---|---|---|---|
| PVA (kg) | 1 kg | 0 | 0.01 |
| H₂O (L) | 1 L | 1 | 0 |

Phase 3 is the nanoemulsion formation process and includes the addition of the internal phase into the continuous phase while stirring and the quantities for 1 L nanoemulsion synthesis process are shown in Table 14.

TABLE 14
Nanoemulsion synthesis
NANOEMULSION SYNTHESIS

| Stream no. | Reagents (L) | Req. Qty (L) |
|---|---|---|
| 1 | PLGA Sol. | 0.1 |
| 2 | PBS Sol. | 0.3 |
| 3 | 1% PVA Sol. | 0.3 |

### 6.3. Phase 4 Preparations and Costing

The gelation phase is the preparation of the viscous mixture comprising of xanthan gum, glycerol and citric acid in water.

TABLE 15
Gelation phase
STREAM 4: GEL FORMULATION

| | | Required Qty/L- Gel | |
|---|---|---|---|
| Reagents | Unit | Liquid stream (L) | Solid Stream (kg) |
| Xanthan gum | 1 kg | 0 | 0.0007 |
| (kg) | | | |
| Glycerol (L) | 1 L | 0.067 | 0 |
| Citric Acid | 1 kg | 0 | 0.005 |
| (kg) | | | |
| H₂O (L) | 1 L | 1 | 0 |

### 6.4. Phase 5 Preparations and Costing

The fusion phase is the final process where the nanoemulsion is added into the viscous gel to produce the nanoemulsion hand sanitizer with Table 16 below showing all streams to produce 1 L of nanoemulsion hand sanitizer.

TABLE 16
Streams to produce the nanoemulsion hand sanitiser

| Stream | 1 | 2 | 3 | 4 | 5 |
|---|---|---|---|---|---|
| Phase | Liquid | Liquid | Liquid | Liquid | Liquid |
| Reaction | R101 | R102 | R103 | R105 | R106 |
| tank | | | | | |
| Solution | PLGA Sol. | PBS Sol. | PVA | Gel | Nanoemulsion |
| | | | Sol. | Formulation | |
| Composition | Acetone (L) | NaCl (kg) | PVA | Xanthan Gum | PLGA Sol. |
| | | | (kg) | (kg) | |
| | Ethanol (L) | KCl (kg) | H₂O (L) | Glycerol (L) | PBS Sol. |
| | Tween 80 | NaH₂PO₄ | — | Citric acid | 1% PVA Sol. |
| | (L) | (kg) | | (kg) | |
| | PLGA (kg) | KH₂PO₄ | — | H₂O (L) | — |
| | | (kg) | | | |
| | — | H₂O (L) | — | — | — |

TABLE 17
Compositions for 1L nanoemulsion hand sanitiser
HAND SANITISER

| Formulation | % Composition | Vol Composition (L) |
|---|---|---|
| Xanthan Gum Gel | 99 | 0.99 |
| Nanoemulsion | 1 | 0.01 |

### 7. Conclusion

One of the most currently recommended non-pharmaceutical strategies consists of using 70-95% alcoholic sanitizers in order to control the infection from coronavirus. Frequent usage of such solutions with high content of alcohol (ethyl alcohol, isopropanol or n-propanol) poses harmful effects to humans and often to children below 12 years. One of the long-term expected effects is the depletion of the first line of defence of the skin in fighting the infection (i.e. microbiome as well as the lipid layer), hence making freeways for any attack by microorganisms.

It appears critical that non-alcoholic hand sanitizers are needed to curb the infection from SARS-CoV-2 while preventing the exposure to other microbial species that are able to permeate through the skin if the microbiome is damaged by alcohol-based sanitizers.

The applicant has therefore successfully developed non-alcoholic water-based antimicrobial hand sanitizers, containing safe polymeric compounds together with amphiphilic fatty acids incorporating one or more actives selected from cyanovirin-N, cannabidiol and palmitic acid. These actives were used either individually or in combination, and they have shown strong binding to a pseudo-SARS-CoV-2 virus, resulting in its complete inhibition or de-activation at a relatively low concentration.

The systems are shown to be safe following an in vitro cytotoxicity test. A small-scale irritancy patch test on human skin for 20 individuals was conducted by an accredited laboratory and it resulted in a non-irritant outcome. The system is cost-effective and easy to scale up.

### REFERENCES

Bergamaschi M M, Costa Queiroz R H, Crippa J A S and Zuardi A W. 2011. Safety and side effects of Cannabidiol, a Cannabis sativa constituent. Current Drug Safety, 6: 1-13
Bruni N, Pepa C D, Oliaro-Bosso S, Pessione E et al. 2018. Cannabinoid delivery systems for pain and inflammation treatment. Molecules, 23: 2478; doi:10.3390/molecules23102478
Choi et al. 2008. Cannabidiol Induces Cytotoxicity and Cell Death via Apoptotic Pathway in Cancer Cell Lines. Biomolecules & Therapeutics, 16: 87-94.
Dey B., Lerner D. L., Lusso P., Boyd M. R., Elder J. H. and Berger E. A. 2000. Multiple antiviral activities of Cyanovirin-N: Blocking of human immunodeficiency virus type 1 gp120 Interaction with CD4 and co-receptor and inhibition of diverse enveloped viruses. J. Virol., 74: 4562-4569.
Dixit, A., Pandey, P., Mahajan, R. and Dhasmana, D. C. 2014. Alcohol based hand sanitizers: Assurance and apprehensions revisited. Res. J. Pharm. Biol. Chem. Sci., 5: 558-563.
Fasinu P.S. et al. 2016. Current Status and Prospects for Cannabidiol Preparations as New Therapeutic Agents. Pharmacotherapy, 36(7): 781-96
Jensen S. M. R., Ruscetti F. W., Rein A., Bertolette D. C., Saucedo C. J., O'Keefe B. R. and Jones K. S. 2014. Differential Inhibitory Effects of Cyanovirin-N, Griffithsin, and Scytovirin on Entry Mediated by Envelopes of Gammaretroviruses and Deltaretroviruses. J. Virol., 88: 2327-2332.
Jing J. L. J., Yi T. P., Bose R. J. C., McCarthy J. R., Tharmalingam N. and Madheswaran T. 2020. Hand sanitizers: A review on formulation aspects, adverse effects, and regulations. Int. J. Environ. Res. Public Health, 17: 3326-3343.
Kinane D F, Stathopoulou P G and Papapanou P N. 2017. Periodontal diseases. Nature Reviews—Disease Primers, 3: 17038. doi:10.1038/nrdp.2017.38
Martinenghi L. D., Jønsson R., Lund T. and Jenssen H. 2020. Isolation, Purification, and Antimicrobial Characterization of Cannabidiolic Acid and Cannabidiol from Cannabis sativa L. Biomolecules, 10: 1-15.
Mitchell C. A., Ramessar K. and O'Keefe B, R. 2017. Antiviral lectins: Selective inhibitors of viral entry. Antivir. Res., 142: 37-54.
Mosmann, T. 1983. Rapid colorimetric assay for cellular growth and survival: Application to proliferation and cytotoxicity assays. Journal of Immunological Methods, 65 (1-2): 55-63
Murthy, S. and H. Shivakumar. 2010. Topical and transdermal drug delivery. Handbook of Non-Invasive Drug Delivery Systems, 1-36.
Nakano Y, Tajima M, Sugiyama E, Sato V H and Sato Hitoshi. 2019. Development of a novel nanoemulsion formulation to improve intestinal absorption of Cannabidiol. Medical Cannabis and Cannabinoids, 2: 35-42
Nguyen M. T., Hanzelmann D., Hartner T., Peschel A. and Gotz F. 2015. Skin-specific unsaturated fatty acids boost the Staphylococcus aureus innate immune response. Infect Immun., 84: 205-215.
Pawan P., Mayur P. and Ashwin S. 2011. Role of natural polymers in sustained release drug delivery system; International Research Journal of Pharmacy, 2: 6-11
Ramer R. and Hinz B. 2017. Chapter twelve—Cannabinoids as anticancer drugs. Advances in Pharmacology, 80: 397-436;
Ravi et al. 2017: Comparative pharmacokinetic evaluation of Iopinavir and lopinavir-loaded solid lipid nanoparticles in hepatic impaired rat model. Journal of Pharmacy and Pharmacology, 69: 823-833
Sattar, S. A. Microbicides and the environmental control of nosocomial viral infections. J. Hos. Infect. 56 (2004) 64-69.
Seo G., Lee G., Kim M. J., Baek S.-H., Choi M., Ku K. B., Lee C.-S., Jun S., Park D., Kim H. G., Kim S.-J., Lee J. -O., Kim B. K, Park E. C. and Kim S. I. 2020. Rapid detection of COVID-19 causative virus (SARS-CoV-2) in human nasopharyngeal swab specimens using field-effect transistor-based biosensor. ACS Nano, 14: 5135-5142
Wassmann C. S., Hojrup P. and Klitgaard J. K. 2020. Cannabidiol is an effective helper compound in combination with bacitracin to kill Gram-positive bacteria. Sci. Rep., 10: 4112.
Wang et al. 2020. Remdesivir and chloroquine effectively inhibit the recently emerged novel coronavirus (2019-nCoV) in vitro. Cell Research, 30: 269-271.
Shrivastava A, Kuzontkoski P M, Groopman J E and Prasad A. 2011. Cannabidiol induces programmed cell death in breast cancer cells by coordinating the cross-talk between apoptosis and autophagy. American Association for Cancer Research, 10 (7): 1161-72
Zhang X, Qin Y, Pan Z, Li M, et al (b). 2019. Cannabidiol induces cell cycle arrest and cell apoptosis in human gastric cancer SGC-7901 cell. Biomolecules 9, 302
Zhang Y, Ge Y, Shi W, Hu C, et al (a). 2019. CDS-1548 induces apoptosis in HeLa cells by activating caspase 3. Oncology Letters, 18: 1881-1887

1. A process for producing an alcohol-free, antimicrobial, moisturising hand sanitizer, comprising the steps of:
(i) producing a polymer-based nanoemulsion comprising the steps of:
A.I. co-dissolving at least one hydrophobic active compound selected from cannabidiol (CBD) and palmitic acid (PA), and at least one copolymer in a polar aprotic solvent;
A.II. adding a surfactant to form an organic phase;
A.III. dispensing the organic phase into an aqueous mixture comprising at least one hydrophilic polymer to form a nanoemulsion; and
A.IV. stabilising the nanoemulsion by stirring, or
B.V. dissolving at least one copolymer in a polar aprotic solvent;
B.VI. adding a surfactant to form an organic phase;
B.VII. dispensing the organic phase into an aqueous mixture comprising at least one hydrophilic polymer and at least one hydrophilic active compound selected from cyanovirin-N (CV-N) to form a nanoemulsion; and
B.VIII. stabilising the nanoemulsion by stirring, or
C.V. co-dissolving at least one hydrophobic active compound selected from CBD, PA or both, and at least one copolymer in a polar aprotic solvent;
C.VI. adding a surfactant to form an organic phase;
C.VII. dispensing the organic phase into an aqueous mixture comprising at least one hydrophilic polymer and at least one hydrophilic active compound selected from CV-N to form a nanoemulsion; and
C.VIII. stabilising the nanoemulsion by stirring; and
(ii) producing an aqueous gel-like moisturizing composition comprising the steps of:
D.I. mixing xanthan gum into glycerol by stirring until completely dispersed in the glycerol; and
D.II. adding water and a preservative including citric acid, and stirring to form the aqueous gel-like moisturizing composition; and
(iii) mixing the stabilised nanoemulsion of step (i) to the aqueous gel-like moisturizing composition of step (ii) to form the alcohol-free, antimicrobial, moisturising hand sanitizer.

2. A process for producing an alcohol-free, antimicrobial, moisturising hand sanitizer, consisting of the steps of:
(i) producing a polymer-based nanoemulsion consisting of the steps of:
A.I. co-dissolving at least one hydrophobic active compound selected from cannabidiol (CBD) and palmitic acid (PA), and at least one copolymer in a polar aprotic solvent;
A.II. adding a surfactant to form an organic phase;
A.III. dispensing the organic phase into an aqueous mixture comprising at least one hydrophilic polymer to form a nanoemulsion; and
A.IV. stabilising the nanoemulsion by stirring, or
B.V. dissolving at least one copolymer in a polar aprotic solvent;
B.VI. adding a surfactant to form an organic phase;
B.VII. dispensing the organic phase into an aqueous mixture comprising at least one hydrophilic polymer and at least one hydrophilic active compound selected from cyanovirin-N (CV-N) to form a nanoemulsion; and
B.VIII. stabilising the nanoemulsion by stirring, or
C.V. co-dissolving at least one hydrophobic active compound selected from CBD and PA, and at least one copolymer in a polar aprotic solvent;
C.VI. adding a surfactant to form an organic phase;
C.VII. dispensing the organic phase into an aqueous mixture comprising at least one hydrophilic polymer and at least one hydrophilic active compound selected from CV-N to form a nanoemulsion; and
C.VIII. stabilising the nanoemulsion by stirring; and
(ii) producing an aqueous gel-like moisturizing composition consisting of the steps of:
D.I. mixing xanthan gum into glycerol by stirring until completely dispersed in the glycerol; and
D.II. adding water and a preservative selected from citric acid, and stirring to form the aqueous gel-like moisturizing composition; and
(iii) mixing the stabilised nanoemulsion of step (i) to the aqueous gel-like moisturizing composition of step (ii) to form the alcohol-free, antimicrobial, moisturising hand sanitizer.

3. The process according to either claim 1 or claim 2, wherein the at least one copolymer is selected from the group consisting of poly (lactic-co-glycolic acid), polylactic acid, polyglycolic acid, and poly 8-caprolactone.

4. The process according to either claim 1 or claim 2, wherein the at least one copolymer is poly (lactic-co-glycolic acid.

5. The process according to any one of the preceding claims wherein the polar aprotic solvent is selected from any one or more of ethanol, acetone, and a blend of ethanol and acetone.

6. The process according to claim 5, wherein the polar aprotic solvent is a co-solution of acetone/ethanol (3:1).

7. The process according to any one of the preceding claims wherein the surfactant is selected from any surfactant having a Hydrophile-Lipophile Balance (HLB) value of greater than 10.

8. The process according to claim 7, wherein the surfactant is polysorbate 80.

9. The process according to any one of the preceding claims wherein the at least one hydrophilic polymer is selected from the group consisting of any one or more of polyvinyl alcohol (PVA) and polyethylene glycol (PEG).

10. The process according to claim 9, wherein the at least one hydrophilic polymer is hydrolysed PVA.

11. The process according to any one of the preceding claims wherein the phosphate buffer has a pH of from about 7.2 to about 7.6, including any possible subrange or pH within this range, at 0° C.

12. An alcohol-free, antimicrobial, moisturising hand sanitizer produced according to the process of any one of claims 1 to 11.

13. The hand sanitizer according to claim 12, which contains an active compound selected from any one of:
(a) CV-N, CBD or PA;
(b) both of CV-N and CBD, or both of CBD and PA, or both of CV-N and PA; and
(c) all three of CV-N, CBD and PA.

14. The hand sanitizer according to either claim 12 or 13 for use an antimicrobial agent, including any one or more of an antibacterial, antiviral or antifungal agent.

15. The hand sanitizer according to any one of claims 12 to 14, wherein the polymer-based nanoemulsion comprises:
i. an inner nanoemulsion matrix comprised of a polar aprotic solvent, at least one copolymer and a surfactant:
ii. an outer shell comprising one or more hydrophilic polymers and a buffer; and
iii. one or more of the active compound(s),
wherein where active compound(s) is(are) hydrophobic, the active compound(s) is(are) comprised in the inner nanoemulsion matrix, and wherein the active compound(s) is(are) hydrophilic, the active compound(s) is(are) comprised in the outer shell.

16. The hand sanitizer according to any one of claims 12 to 14, wherein the polymer-based nanoemulsion consists of:
A.a) an inner nanoemulsion matrix consisting of a polar aprotic solvent, at least one copolymer, a surfactant and any one or more active compound(s) selected from CBD and PA; and
A.b) an outer shell consisting of one or more hydrophilic polymers and a buffer, or
B.c) an inner nanoemulsion matrix consisting of a polar aprotic solvent, at least one copolymer, and a surfactant; and
B.a) an outer shell consisting of one or more hydrophilic polymers, a buffer and an active compound selected from CV-N, or
C.a) an inner nanoemulsion matrix consisting of a polar aprotic solvent, at least one copolymer, a surfactant and any one or more active compound(s) selected from CBD and PA; and
C.b) an outer shell consisting of one or more hydrophilic polymers, a buffer and an active compound selected from CV-N.

17. The hand sanitizer according to any one of claims 12 to 16, wherein the aqueous solution of hydrophilic polymers are selected from any one or more of polyvinyl alcohol (PVA) and polyethylene glycol (PEG).

18. The hand sanitizer according to claim 17, wherein the hydrophilic polymer is hydrolysed PVA.

19. The hand sanitizer according to any one of claims 12 to 18, wherein the at least one copolymer is selected from the group consisting of poly (lactic-co-glycolic acid), polylactic acid, polyglycolic acid, and poly ε-caprolactone.

20. The hand sanitizer according to claim 19, wherein the copolymer is poly (lactic-co-glycolic acid).

21. The hand sanitizer according to any one of claims 12 to 20, wherein the polar aprotic solvent is selected from the group consisting of ethanol, acetone and a blend thereof.

22. The hand sanitizer according to claim 21, wherein the polar aprotic solvent is a co-solution of acetone/ethanol (3:1).

23. The hand sanitizer according to any one of claims 12 to 22, wherein the surfactant has a Hydrophile-Lipophile Balance (HLB) value of greater than 10, including polysorbate 80.

24. The hand sanitizer according to any one of claims 12 to 23, wherein the buffer is phosphate buffered saline (PBS).

25. A method for sanitising hands and optionally surfaces with the alcohol-free, antimicrobial, moisturising hand sanitizer according to any one of claims 12 to 24.

26. A method for inhibition of microbes, including bacteria, viruses and fungi, with the use of the alcohol-free, antimicrobial, moisturising hand sanitizer according to any one of claims 12 to 24.

27. The method according to either claim 25 or 26 wherein the microbes are viruses, including any one or more of HIV-1 or coronaviruses including SARS-CoV-2 or MERS-CoV.

28. Use of the alcohol-free, antimicrobial, moisturising hand sanitizer according to any one of claims 12 to 24 in a method of sanitising hands and/or inhibition of microbes, including bacteria, viruses and fungi.

29. The use according to claim 28 wherein the microbes are viruses, including any one or more of HIV-1 or coronaviruses including SARS-CoV-2 or MERS-CoV.
